(19) United States
(12) Patent Application Publication
(10) Pub. No.: US 2006/0270610 A1
(43) Pub. Date: Nov. 30, 2006

(54) Novel compounds for hypoxic cell therapy and imaging

(76) Inventors: Leonard Wiebe, Edmonton (CA); Alexander J.B. McEwan, Edmonton (CA); Piyush Kumar, Edmonton (CA)

Correspondence Address: Craig K. Sherburne, #35, 1011 Canterbury Drive SW, Calgary, AB, T2W 2S8, CA

(21) Appl. No.: 11/391,407

(22) Filed: Mar. 29, 2006

Related U.S. Application Data
(60) Provisional application No. 60/665,876, filed on Mar. 29, 2005.

(51) Int. Cl. A61K 31/7052; C07H 17/02

(52) U.S. Cl. 514/23; 536/17.4

(57) ABSTRACT

The present invention provides for compounds suitable for therapeutic treatment of hypoxic tissues, particularly for application in radiotherapy, chemosensitization, radiosensitization. The present invention further provides for compounds suitable for radioimaging of hypoxic cells.

### RELATED APPLICATION

This application claims the benefit of U.S. Provisional Application Ser. No. 60/665,876, filed Mar. 29, 2005, under 35 U.S.C. 119(e). The entire disclosure of the prior application is hereby incorporated by reference.

### FIELD OF THE INVENTION

The present invention relates to the fields of human therapeutics, diagnostics, radioimaging and chemotherapy.

### BACKGROUND OF THE INVENTION

All of the publications, patents and patent applications cited within this application are herein incorporated by reference in their entirety to the same extent as if the disclosure of each individual publication, patent application or patent was specifically and individually indicated to be incorporated by reference in its entirety.

Decreased oxygen levels in tumor cells increases their resistance to the damaging effects of ionizing radiations (Tomlinson R. H., et al Br J Cancer 9:539 (1955)), an effect that is thought to greatly reduce the efficacy of conventional low linear energy transfer radiation (e.g. X-ray) therapies (Brown J. M. Cancer Res 59:5863 (1999)). 2-nitroimidaole(azomycin) nucleosides are highly diffusible radiosensitizers that readily permeate hypoxic tissues, where they are bioreductively activated by single electron transfer and subsequently selectively bound as molecular adducts within viable hypoxic cells. The reversibility of this single electron reduction in the presence of oxygen limits adduct formation to cells that are pathologically hypoic (Bigalow, J. E. et al Biochem Pharmac 35:77 (1986)).

This oxygen-dependent selectivity forms the basis for non-invasive (imaging) diagnosis of a hypoxic region with radiolabelled nitroimidazoles. (Chapman J. D. et al Cancer 43:456 (1981); Adams, G. E. Radia Res 67:9 (1976)). In the past, a number of radioiodinated azomycin α-nucleosides have been synthesized and explored to detect and monitor regional hypoxia (Jette D. C. et al Radiat Res 105:169 (1986); Wiebe L. I in “Nuclear Medicine in Clinical Oncology” 402 (1986)). Of these, 1-α-D-(5-dexoy-5-iodorabinofuranosyl)-2-nitroimidazole (IAZA) has been widely studied and clinically used in a variety of pathologies involving tissue hypoxia (Parliament M. B. et al Br J Radiol 65:90 (1991); Groshar D. Nucl Med 34:885 (1993); Urtasun R. C. et al Br J Cancer 74:S209 (1996); Al-Arafaj A. et al Europ J Nucl Med 21:1338 (1994); McEwan A. J. B. et al J Nucl Med 38:300 (1997); Vinjamuri S. Clin Nucl Med 24:8912 (1999)).

Nitroimidazole radiosensitizers were used to overcome the ‘oxygen effect’ through an oxygen mimicking processes that results in radiosensitization through selective bioactivation and consequent binding (adduct formation) to tissue components (Adams G. E. et al Int J Radiat Biol 15:457 (1969)). In this process, the first-electron reduction is reversible in the presence of oxygen, therefore, the ultimate degree of binding is dependent on the absence (low concentration) of oxygen. Reducing equivalents (electrons) for this process are metabolically-derived (Bigalow J. E. et al Biochem Pharmac 35:77 (1986)), and therefore the adduct-based accumulation of azomycins is restricted to viable tissue that is O₂-deficient, with no accumulation in necrotic cells and little accumulation and low toxicity in most normally-oxygenated cells.

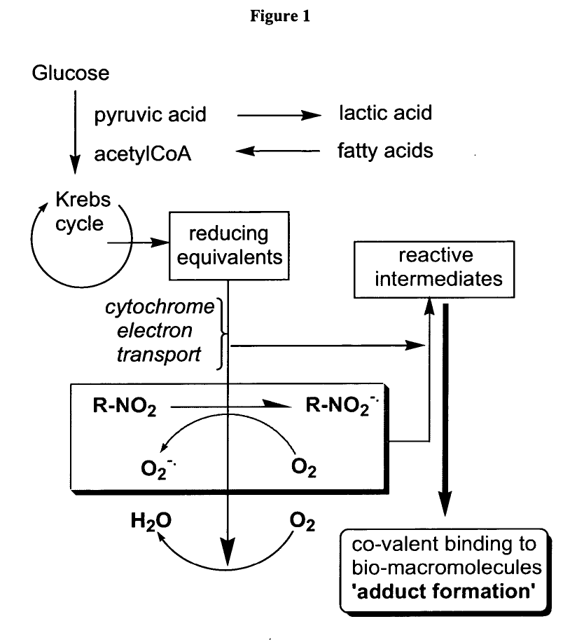

Flavin-dependent cytochrome P450 reductase and related enzymes, including xanthine and aldehyde oxidases, and quinone oxidase are thought capable of carrying out this reductive bioactivation. Electron affinity of the substrate (e.g. azomycins) dictates both sensitivity to O₂ and toxicity of the tracer. If the first, single-electron, reduction potential (first electron reduction potential, E¹₇) approaches that of O₂ (−155 mV), then selectivity for hypoxia will be diminished; if it is not sufficiently electron-affinic (E¹₇<−450 mV), then sensitivity will be lost. This step is critical, since it is reversible by O₂ and is therefore responsible for selective binding to only those tissues that are O₂ deficient. The E¹₇'s of most 2-nitroimidazoles lie around −390 mV, an electron affinity considered to be optimal for selectivity and sensitivity (Adams G. E. et al Radiat Res 67:9 (1976)). A schematic representation of these processes is depected in FIG. 1.

Thus, hypoxia-sensitive radiopharmaceuticals are reduced by electrons produced during glycolysis and by the Krebs Cycle. Flavin-dependent cytochrome P450 reductase, and xanthine-, aldehyde- and quinone-oxidases are among the activating (i.e. reductive) enzymes. (Biaglow J. E. et al Biochem Pharmac 35:77 (1986)). The cell must be viable, even if oxidatively quiescent, to carry out this function, a property which discriminates between dead and stunned but salvageable tissue.

Hypoxic tissue is also ischemic. It is therefore equally important that the radiosensitizer is a facile tissue permeant, meaning that the molecules must be moderately lipophilic. The ability of radiosensitizers (and any compound that is not actively transported) to move freely across cell membranes is based on their lipophilicity (Brown J. M. et al Radiat Res 82:171 (1980)). However, if lipophilicity is too high, they will dissolve in lipoidal tissues and exhibit selective toxicities (e.g. neuropathies). If they are too hydrophilic, they will not diffuse readily through cell membranes. Moreover, hydrophilic compounds tend to be cleared very rapidly via the kidney, severely reducing the amount of tracer available for bioreductive activation and hypoxia-dependent binding.

The limitations of the halogenated azomycin compounds, in being transported into the cell and in establishing therapeutic and diagnosticly relevant residence time are known in the art. It is therefore an object of the present invention to describe a class of compounds capable of transport into a cell through equilabrative and/or concentrative means.

It is a further object of the present invention to describe a class of compounds capable of increased cellular residence time.

It is a further object of the present invention to describe a class of compounds capable of increased tumor specificity.

It is a further object of the present invention to describe a class of comopunds capable of increased therapeutic effect.

### SUMMARY OF THE INVENTION

In another embodiment, the present invention provides for compounds suitable for diagnostics, radiotherapy, chemotherapy, radiosensitization and chemosensitization of hypoxic cells; said compounds selected from the group comprising (together “Compound(s) of the Present Invention”)

1-β-D-(Substituted pentosyl/hexosyl)-2-nitroimidazoles and 1-α-D-(Substituted furanosyl/hexopyranosyl)-2-nitroimidazoles, more particularly described as:

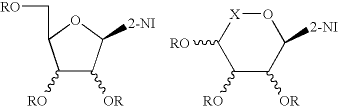

1-β-D-(2,3,5/2,3,4-Tri-O-Substituted furanosyl/hexopyranosyl)-2-nitroimidazoles;

wherein

R═H, Ac, Bz, Piv, any halogen, TIPS, TBDPS, TBDMS, SO₂R₁;

R₁═CH₃, toluyl, CF₃, p-nitrobenzene and any other Leaving Group;

2-NI═

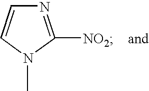

X═—CH₂, or —CHCH₂OH

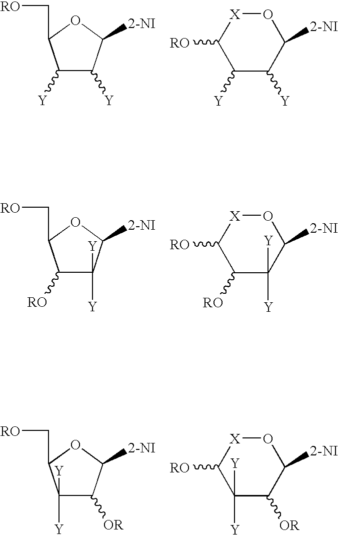

1-β-D-[(2/3-Substituted) or (2,3-disusbstituted) or (2,2-disubstituted) or (3/3-disubstituted) furanosyl/hexopyranosyl)-2-nitroimidazoles;

Wherein

Y═—H, -D, -T, —OH, —F, ¹⁸F, —Br, ^{75/76/77}Br, —Cl, —I (except at 2′-arabinose position), ¹²⁵I, ¹²³I, ¹³¹I, ¹²⁴I, —At and ²¹¹At,;

R═—H;

2-NI═

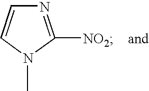

X═—CH₂, or —CHCH₂OH

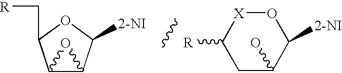

1-β-D-[(2/3-Epoxy)-5-susbstituted furanosyl/hexopyranosyl)-2-nitroimidazoles;

Wherein

R═OH, OAc, OBz, OPiv, any halogen or OSO₂R₁ substituents at these positions;

R₁═CH₃, toluyl, CF₃, p-nitrobenzene and any other Leaving Group.

2-NI═

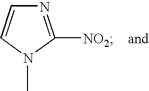

X═—CH₂, or —CHCH₂OH

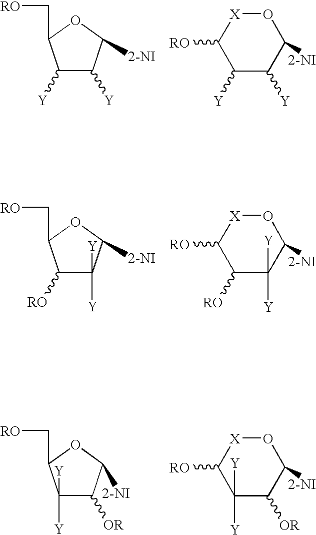

1-α-D-[(2/3-Substituted) or (2,3-disusbstituted) or (2,2-disubstituted) or (3/3-disubstituted) furanosyl/hexopyranosyl)-2-nitroimidazoles.

Wherein

Y═H, D, T, OH, F, ¹⁸F, Br, ^{75/76/77}Br, Cl, ^{34m/34/36}Cl, I, ¹²⁵I, ¹²³I, ¹³¹I, ¹²⁴I, At and ²¹¹At;

R═H;

2-NI═

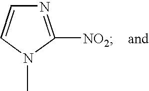

X═—CH₂, or —CHCH₂OH

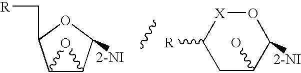

1-α-D-[(2/3-Epoxy)-5-susbstituted furanosyl/hexopyranosyl)-2-nitroimidazoles.

Wherein

R═OH, OAc, OBz, OPiv, every halogen and OSO₂R, substituents;

R₁═CH₃, toluyl, CF₃, p-nitrobenzene and any other Leaving Group;

2-NI═

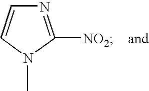

X═—CH₂, or —CHCH₂OH

The present invention further provides for said Compound of the Present Invention to contain a radionuclide suitable for radiotherapy, said radionuclide selected from the group consisting of ²¹¹At, ¹²⁵I, ¹³¹I, ⁷⁶Br, ⁷⁷Br, ⁸²Br, ^{34m}Cl and ²⁴Cl;

The present invention further provides for use of said compound in association with radiotherapy so as to render a hypoxic cell more susceptible to radiotherapy.

The present invention further provides for use of said compound in association with chemotherapy so as to render a hypoxic cell more susceptible to chemotherapy.

The present invention further provides for the use of said compound for radioimaging hypoxic cells wherein the halogen in said compound is replaced with a radionuclide suitable for radioimaging.

The present invention further provides for compounds suitable for radiotherapy, chemotherapy, radiosensitization and chmeosensitization of hypoxic cells; said compounds selected from the group consisting of:
1-β-D-[3-deoxy-3-fluoroxylofuranosyl]-2-nitroimidazole;
1-β-D-[3-deoxy-3chloroxylofuranosyl]-2-nitroimidazole;
1-β-D-[3-deoxy-3-bromoroxylofuranosyl]-2-nitroimidazole;
1-β-D-[3-deoxy-3-fluororibofuranosyl]-2-nitroimidazole;
1-β-D-[3-deoxy-3-chlororibofuranosyl]-2-nitroimidazole;
1-β-D-[3-deoxy-3-bromoribofuranosyl]-2-nitroimidazole;
1-β-D-[2-deoxy-2-fluororibofuranosyl]-2-nitroimidazole;
1-β-D-[2-deoxy-2-chlororibofuranosyl]-2-nitroimidazole;
1-β-D-[2-deoxy-2-bromoribofuranosyl]-2-nitroimidazole;
1-β-D-[3-deoxy-3-fluoroarabinofuranosyl]-2-nitroimidazole;
1-β-D-[3-deoxy-3-chloroarabinofuranosyl]-2-nitroimidazole;
1-β-D-[3-deoxy-3-bromoarabinofuranosyl]-2-nitroimidazole;
1-β-D-[2-deoxy-2-fluoroarabinofuranosyl]-2-nitroimidazole;
1-β-D-[2-deoxy-2-chloroarabinofuranosyl]-2-nitroimidazole;
1-β-D-[2-deoxy-2-bromoarabinofuranosyl]-2-nitroimidazole;
1-β-D-[3,2-dideoxy-3-fluoroarabinofuranosyl]-2-nitroimidazole;
1-β-D-[3,2-dideoxy-3-chloroarabinofuranosyl]-2-nitroimidazole;
1-β-D-[3,2-dideoxy-3-bromoarabinofuranosyl]-2-nitroimidazole;
1-β-D-[3,2-dideoxy-3-fluoroarabinofuranosyl]-2-nitroimidazole;
1-β-D-[3,2-dideoxy-3-chloroarabinofuranosyl]-2-nitroimidazole;
1-β-D-[3,2-dideoxy-3-bromoarabinofuranosyl]-2-nitroimidazole;
1-β-D-[2,3-dideoxy-2-fluoroarabinofuranosyl]-2-nitroimidazole;
1-β-D-[2,3-dideoxy-2-chloroarabinofuranosyl]-2-nitroimidazole;
1-β-D-[2,3-dideoxy-2-bromoarabinofuranosyl]-2-nitroimidazole;
1-β-D-[2,3-dideoxy-2-fluororibofuranosyl]-2-nitroimidazole;
1-β-D-[2,3-dideoxy-2-chlororibofuranosyl]-2-nitroimidazole;
1-β-D-[2,3-dideoxy-2-bromoribofuranosyl]-2-nitroimidazole;
1-β-D-[2-deoxy-2-keto-arabinofuranosyl]-2-nitroimidazole;
1-β-D-[3-deoxy-3-keto-arabinofuranosyl]-2-nitroimidazole; and
1-β-D-[2,3-dideoxy-2,3-epoxyarabinofuranosyl]-2-nitroimidazole.

In another aspect, the present invention provides for methods of treating a patient suffering from cancer comprising administration of an effective amount of at least one Compound of the Present Invention followed by radiotherapy.

In the case of diagnostic applications, increased localization of a Compound of the Present Invention in hypoxic cells as compared to less hypoxic or oxic cells, the Compound of the Present Invention labelled with a radioisotope capable of being imaged, facilitates the detection of the hypoxic cells.

In the case of radiotherapy applications, increased localization of a Compound of the Present Invention in hypoxic cells as compared to less hypoxic or oxic cells, the Compound of the Present Invention radioactive as a result of the compound being radiolabelled, permits the product to preferentially accumulate in hypoxic cells, thus facilitating radiotherapeutic effects directed specifically at hypoxic cells.

In a first embodiment relating to diagnostic applications of the invention, the invention comprises a method for monitoring hypoxic cells within a population of less hypoxic or oxic cells, comprising the steps of administering to the cells an effective amount of a labelled Compound of the Present Invention so that the labelled compound accumulates preferentially in hypoxic tissues and then detecting labelled compound. In this first embodiment, the invention also comprises the use of labelled Compounds of the Present Invention in performing this diagnostic method.

In a preferred diagnostic method, the method for monitoring hypoxic cells throughout the population of cells is comprised of the following steps:

(a) administering to the cells an effective amount of at least one labelled Compound of the Present Invention;

(b) waiting a period of time such that a substantial amount of the at least one labelled Compound of the Present has been expelled from less hypoxic or oxic cells and such that a detectable amount of the at least one labelled Compound of the Present Invention remains within hypoxic cells; and

(c) determining the extent and location of hypoxic cells throughout the population of cells by detecting the at least one labelled Compound of the Present Invention.

The labelled Compound of the Present Invention is preferably radiolabelled, but any other form of labelling which facilitates detection of the labelled Compound of the Present Invention may also be suitable. One non-limiting example is the inclusion of isotopes in the Compound of the Present Invention which are identifiable using Nuclear Magnetic Resonance or Magnetic Resonance Imaging.

In the case of diagnostic applications, preferential localization of the Compound of the Present Invention in hypoxic cells as compared less hypoxic or oxic cells permits the compound of the Present Invention to accumulate in hypoxic cells in order to facilitate the detection of the labelled product in those cells.

Accordingly, following the administration of an effective amount of the labelled Compound of the Present Invention to a patient such that the labelled Compound of the Present Invention accumulates preferentially in hypoxic tissues, the diagnostic method includes waiting a period of time such that a substantial amount of the labelled Compound of the Present Invention has been expelled from the less hypoxic or oxic cells and such that a detectable amount of the labelled Compound of the Present Invention remains within hypoxic cells. As there is a preferential accumulation of the Compound of the Present Invention in cells experiencing more hypoxic conditions than those of lesser hypoxic or oxic conditions, this is a matter of waiting a period of time allowing the preferred amount of clearance from lesser hypoxic cells or oxic cells as compared to hypoxic cells. One skilled in the art will be capable of determining the appropriate amount of time with observation and as a function of administered dose, patient weight, patient age, patient sex, and suspected hypoxic cell location in the body.

The period of time for waiting, prior to performing the step of determining the extent and location of hypoxic cells throughout the population of cells by detecting the labelled Compound of the Present Invention, will be determined or selected depending upon a number of various factors including the properties of each of the labelled Compound of the Present Invention. For instance, the rate of expulsion or clearance of each of the labelled Compound of the Present Invention in hypoxic cells compared to cells of lesser hypoxia or cells in oxic conditions. The time period is selected to achieve a balance between the amount of the labelled Compound of the Present Invention present in the hypoxic cells and the amount of the Compound of the Present Invention present in the cells of lesser hypoxia or oxic conditions at the time of detecting the labelled Compound of the Present Invention. First, the amount of the labelled Compound of the Present Invention is preferably minimized in order to enhance or increase the accuracy of the diagnostic method as the presence of significant or substantial amounts of the labelled Compound of the Present Invention may interfere with the detection of the labelled Compound of the Present Invention localized in hypoxic cells. For instance, in radiolabelling of the Compound of the Present Invention, radioimaging may be unable to distinguish between the presence of the labelled Compound of the Present Invention in hypoxic cells as compared with lesser hypoxic or oxic cells if too much labelled Compound of the Present Invention is administered. Second, the amount of the labelled Compound of the Present Invention within the hypoxic cells is preferably maximized to facilitate the detection of the labelled Compound of the Present Invention in hypoxic cells as compared to cells of lesser hypoxia or cells in oxic conditions and to also enhance or increase the accuracy of the diagnostic method.

Most preferably, the labelled Compound of the Present Invention rate of clearance in hypoxic cells compared to cells of lesser hypoxia or cells in oxic conditions is such that following the passage of a determined or selected period of time, all or substantially all of the labelled Compound of the Present Invention has been expelled from the cells of lesser hypoxia or cells in oxic conditions while all or substantially all of the labelled Compound of the Present Invention remains within the hypoxic cells. In other words, the expulsion of the labelled Compound of the Present Invention from cells of lesser hypoxia or cells in oxic conditions and the expulsion of the labelled Compound of the Present Invention from hypoxic cells do not overlap such that the expulsion of the labelled Compound of the Present Invention from cells of lesser hypoxia or cells in oxic conditions is complete or substantially complete prior to the commencement of any expulsion or any substantial expulsion of the labelled Compound of the Present Invention from hypoxic cells.

However, the relative rates of clearance may provide for an overlap of the expulsion of the labelled Compound of the Present Invention in hypoxic cells compared to cells of lesser hypoxia or cells in oxic conditions. In this case, the period of time is selected or determined according to the desired degree of accuracy or the desired statistical significance of the diagnostic test results as discussed above. As indicated, the period of time is selected so that preferably a substantial amount of the labelled Compound of the Present Invention has been expelled. For a substantial amount to be expelled, any remaining labelled Compound of the Present Invention in cells of lesser hypoxia or cells in oxic conditions is not enough to significantly interfere with the detection of the labelled Compound of the Present Invention in hypoxic cells and is such that the diagnostic test results achieve the desired degree of accuracy or statistical significance. The period of time is also selected so that a detectable amount of the labelled Compound of the Present Invention remains within the hypoxic cells. A detectable amount is present if there is a sufficient amount to permit effective detection according to the selected detection method or process and such that the diagnostic test results achieve the desired degree of accuracy or statistical significance. For instance, where radiolabelling and radioimaging are used, a sufficient amount of the labelled Compound of the Present Invention must remain in the hypoxic cells to provide adequate signal measurement.

Once this period of time has passed, the extent and location of the hypoxic cells throughout the population of lesser hypoxic or oxic cells is determined by detecting the labelled Compound of the Present Invention. The determination of the extent and location of the Compound of the Present Invention in the cells provides for or permits the monitoring of regions of hypoxia. These regions of hypoxia correlate with the presence of a collection of cancerous cells or tumors.

The method of detection is selected according to the type or manner of the labelling of the Compound of the Present Invention. However, in the preferred embodiment, the labelled Compound of the Present Invention is radiolabelled and the detection is performed using nuclear medicine imaging techniques.

In a second embodiment relating to radiotherapy applications of the invention, the invention comprises a method of radiotherapy for use with a population of hypoxic cells, comprising the step of administering to the cells an effective radiotherapeutic dose of a radiolabelled Compound of the Present Invention so that the radiolabelled Compound of the Present Invention becomes preferentially localized within hypoxic cells. In this second embodiment, the invention also comprises the use of radiolabelled Compound of the Present Invention in performing this radiotherapy method. The substituents for the specific preferred radiolabelled Compound of the Present Invention for use with this radiotherapy applications are the same as for the diagnostic method of the invention, except that the radiolabelled compounds are selected from the group consisting of, but not limited to, ¹²³I, ¹²⁵I and ¹³¹I.

In a third embodiment relating to chemotherapy applications of the invention, the invention comprises a method of chemotherapy for use with a population of cells suspected of containing hypoxic cells, comprising the step of administering to the cells an effective chemotherapeutic amount of a Compound of the Present Invention wherein the Compound of the Present Invention is cytotoxic or cytostatic. In this embodiment, the invention also comprises the use of Compound of the Present Invention in performing this chemotherapy method.

The appropriate time interval or period of time between injection of the radiolabelled Compound of the Present Invention and imaging depends on, amongst other factors, the half-life of the radiolabelled Compound of the Present Invention, the rate of clearance of the radiolabelled Compound of the Present Invention in hypoxic cells compared to and the rate of clearance of the radiolabelled Compound of the Present Invention in cells of lesser hypoxia or cells in oxic conditions. Thus, the time period must be particularly determined or selected for each specific labelling and dosing paradigm. A time period of 1.5-24 h is most common, with the shorter periods used for ¹⁸F imaging and the longer times for radiolabels like ¹²³I. After the appropriate time period, retained radioactivity will be due to the Compound of the Present Invention in hypoxic cells. Optimal times are selected to provide best image contrast, that is, the time when excretion of the radiolabelled Compound of the Present Invention in cells of lesser hypoxia or cells in oxic conditions is complete or substanially complete, and sufficient radiolabelled Compound of the Present Invention in hypoxic cells remains for adequate signal measurement. A positive image will show uptake of radioactivity in a region, which reflects proof of cellular hypoxia (i.e. measurement by imaging). Nuclear medicine imaging techniques, including planar (2-dimensional), positron emission tomography (PET) and single photon emission tomography (SPECT) imaging, and their interpretations, are known to practitioners versed in the art.

Those skilled in medical radiotherapeutic methods and uses will be able to calculate a suitable effective dose of the radiolabelled Compound of the Present Invention for human or other uses based on their experience with other compounds carrying similar radiolabels. However, as indicated previously, when the radiolabelled Compound of the Present Invention is used for diagnostic purposes, as small a dosage as possible should be used in order to minimize any toxicity to the population of cells or surrounding tissue. When using the compound for radiotherapeutic purposes, an effective radiotherapeutic dose of the radiolabelled Compound of the Present Invention must be used. Typically, the dosage of the radiolabelled Compound of the Present Invention for therapeutic purposes will be greater than that used for diagnostic purposes in order to achieve the desired radiotherapeutic effect. When used on cancerous cells, the desired radiotherapeutic effect will be a cytotoxic or cytostatic effect on the cells in which the radiolabelled Compound of the Present Invention is present. For use as a radiosensitizer, one skilled in the art will recognize that a dosage of Compound of the Present Invention administered will be that which achieves an increase in therapeutic effect of the radiation when the patient is administered with a Compound of the Present Invention, as compared to a patient in which a Compound of the Present Invention is not administered. One skilled in the art will recognize that administration of at least one Compound of the Present Invention can result in an increased therapeutic kill of hypoxic cells, including cancerous or tumor cells, with a given radiation dose, or alternatively reduce the radiation dose utilized to effect a therapeutic kill of hypoxic cells, including cancerous or tumor cells.

The accompanying description illustrates preferred embodiments of the present invention and serves to explain the principles of the present invention.

### BRIEF DESCRIPTION OF THE DRAWNINGS

FIG. 1 shows a schematic of the reduction of azomycins under hypoxic conditions;

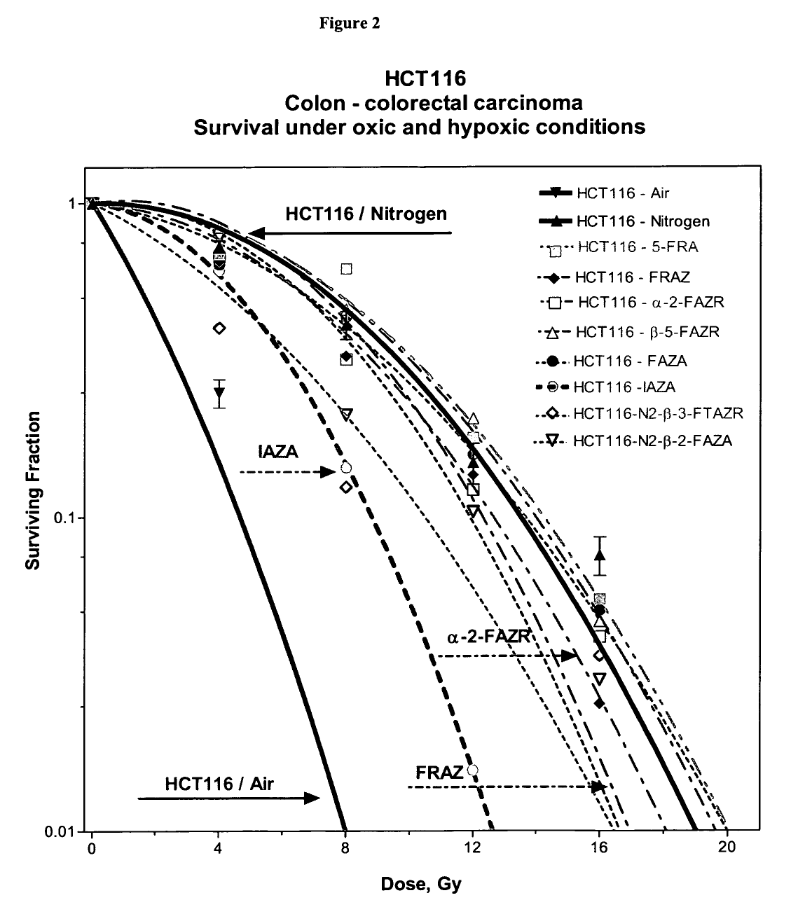

FIG. 2 shows the sensitization of the human colorectal carcinoma cell line HCT116 to radiotherapy with select Compounds of the Present Invention; and

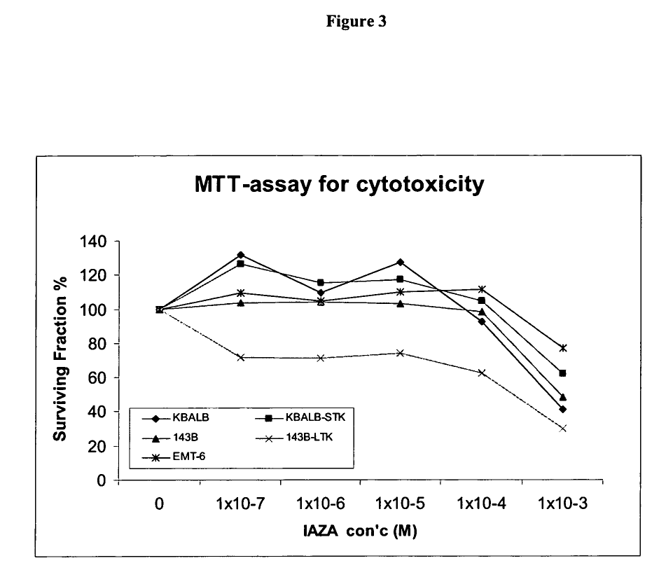

FIG. 3 shows cytotoxicity of β-IAZA in various cell lines as a function of concentration.

### DETAILED DESCRIPTION OF THE INVENTION

Symbols used in this description are explained below.
Symbols Chemical Name
α-AZA 1-α-D-(arabinofuranosyl)-2-nitroimidazole
At Astatine
β-AZA 1-β-D-(arabinofuranosyl)-2-nitroimidazole
Ac Acetyl
Ac2O Acetic anhydride
Br Bromine
Bz Benzoyl
CH2Cl2 Dichloromethane
CH3CN Acetonitrile
Cl Chlorine
CrO3 Chromium trioxide
D Deuterium
DAST Diethylaminosulfurtrifluoride
DMAP N,N-Dimethylaminopyridine
DME Dimethoxyethane
EtOH Ethyl alcohol
EtOAc Ethyl acetate
F Fluorine/Fluoride
HRMS Igh resolution mass spectroscopy
K2CO3 Potassium carbonate
KF Potassium fluoride
MeCN Acetonitrile
MeOH Methanol
N2H2 Hydrazine
NaBDO4 Sodium tetraborodeuteride
NaBTO4 Sodium tetraborotritide
Na2SO4 Sodium sulphate
NH3 Ammonia
NMR Nuclear magnetic resonance
Nosyl p-Nitrobenzenesulfonyl
Piv. Pivaloyl
R4NF Teraalkylammonium fluoride
T Tritium
THF Tetrahydrofuran
TLC Thin layer chromatography
Tosyl Toluenesulfonyl
Triflyl Trifluoromethanesulfonyl
v/v Volume/volume

As used herein “Leaving Group” means the Sulfonyl related leaving groups Methanesulfonyl, substituted methane sulfonyl, trifluoromethanesulfonyl, benzenesulfonyl and all substituted benzenesulfonyl (including but not limited to toluenesulfonyl, nitrobenzenesulfonyl and related compounds); OH, C═O and Halogen related leaving groups including but not limited to Cl, Br and iodine.

An “effective amount” is an amount of a Compound of the Present Invention sufficient to achieve the intended purpose. For example, an effective amount of a Compound of the Present Invention to kill hypoxic or cancerous cells comprising a tumor is an amount sufficient, in vivo to result in an increased killing of hypoxic or cancerous cells as compared to non-hypoxic cells. An effective amount of a Compound of the Present Invention to image hypoxic or cancerous cells comprising a tumor is an amount sufficient, to identify an increased localization of hypoxic or cancerous cells as compared to lesser hypoxic or oxic cells. An effective amount of a Compound of the Present Invention to treat or ameliorate a cancerous disease or condition is an amount of the Compound of the Present Invention sufficient to reduce or remove the symptoms of the cancerous disease or condition. The effective amount of a given Compound of the Present Invention will vary with factors such as the nature of the agent, the route of administration, the size and species of the animal or patient to receive the therapeutic agent, and the purpose of the administration. The effective amount in each individual case may be determined empirically by a skilled artisan according to established methods in the art and the teachings herein.

Chapman postulated that scintigraphic imaging of tumor hypoxia using gamma-emitting nitroimidazole radiosensitizers could form the basis of a useful predictive assay for radiation therapy planning. (Chapman J. D. et al Brit J Cancer 43:546 (1981)). For nitroimidazole-based radiopharmaceuticals, this means that the sensitizer characteristics already identified have to be adjusted to accommodate design limitations imposed by the radionuclide.

Preliminary in vivo biodistribution studies in a murine tumor model, and pharmacokinetic studies in rats indicated that [³H] 1-α-D-FAZA has biodistribution, tumor uptake and pharmacokinetic properties similar to those of ¹²³I-IAZA, a clinically-proven radiopharmaceutical for SPECT-imaging of hypoxic tissues (Kumar P. et al J Label Comp Radiopharm 42:3 (1999)). In vitro and in vivo comparisons of [18F] 1-α-D-FAZA and [¹⁸F]FMISO indicated that hypoxia-selective uptake similar, with faster clearance of [¹⁸F] 1-α-D-FAZA from blood, viscera and muscle tissue, via the renal system of rats (Sorger D. et al Nucl Med Biol 30:317 (2003)). In three different murine tumor models, tumor:blood ratios were 2-4 times greater for [¹⁸F] 1-α-D-FAZA than [18F]FMISO, an effect attributable to rapid blood clearance of [¹⁸F] 1-α-D-FAZA, since tumor uptake, as a fraction of dose, was similar between these tracers. In a nude mouse model bearing a subcutaneous A431 tumor, tumor-background was 9.3:1 for animals breathing room air, compared to 5.3:1 for animals breathing 100 % O₂, demonstrating the oxygen-sensitivity of [18F] 1-α-D-FAZA binding (Piert M. J Nucl Med 43:278P (2002)).

Initial clinical studies complement animal data, reflecting strong uptake by hypoxic tumor and rapid clearance from the vascular compartment, to provide strong target (tumor) to background contrast, and with little hepatic and gastrointestinal signal. In patients studied sequentially with [¹⁸F] 1-α-D-FAZA, [¹⁸F]FMISO and [¹⁸F]FDG, virtually identical images were obtained. One major difference was the absence of [¹⁸F] 1-α-D-FAZA uptake in normal brain tissue, compared to [¹⁸F]FMISO, which is taken up non-specifically by brain, and [¹⁸F]FDG, which is taken up as a reflection of glucose metabolism in healthy brain (Wiebe L. I. in press).

Previous studies on the synthesis of 1-α-D-[5-deoxy-5-iodoarabinofuranosyl]-2-aminoimidazole (iodo aminoimidazole arabinoside; IAIA), a potential nitroreducrase reduction metabolite of IAZA (Mannan R. H. et al J Nucl Med 32:1764 (1991); Edwards D. I. J Antimicrob Chemother 31:9 (1993)), revealed that IAZA, which had previously been assigned the β-configuration, was actually the α-anomer (Lee H. C. et al Nucleosides & Necleorides 18:1995 (1999)). Furthermore, in vitro studies indicated that IAZA was not transported by the NBMPR (nitrobenzylthioinosine)-sensitive equilibrative nucleoside transporter in erythrocytes (Wange L. et al Unpublished) which was not unexpected given that these transporters handle physiological nucleosides that have the β-nucleoside configuration (Cass C. E. in “Drug Transport in Antimicrobial and Anticancer Chemotherapy” 403 (1995)). Therefore a class of compounds useful for selectively residing in hypoxic cells and actively transported into cells, in the β-nucleoside configuration are defined and disclosed herein.

Nucleoside kinases bioactivate nucleosides for incorporation into DNA and RNA by 5′-phosphorylation. In mammalian cells, four deoxyribonucleoside kinases have been characterized, two of which (thymidine kinase; TK1) and deoxycytidine kinase (dCK) are found in the cytoplasm, whereas thymidine kinase (TK2) and the deoxyguanosine kinase (dGK) are predominantly localized in mitochondria (Amer, E. S. J. et al Pharmacol Ther 67:155 (1995)). These kinases are generally substrate-specific, with specificities governed by the base (pyrimidine or purine), the sugar (deoxyribose and ribose), and the configuration of the glycoside bond at the anomeric carbon (C1′; only β anomer) of the sugar. There are important exceptions for each nucleoside kinase; important examples include phosphorylation of 2′-/3′-fluoro-2-′/3′-ribo/arabinofyranosyl pyrimidine nucleosides (altered sugar) (De Clercq E. Mini Rev Med Chem 2:163 (2002)), and imidocarboxamide ribosides (altered base) like EICAR (Balzarini, J. et al Adv Exp Med Biol 431:723 (1998)). Nucleoside kinases play a crucial role in the chemotherapy of cancer and viral infections. These enzymes catalyze the rate-limiting phosphorylation of the nucleoside-analogue pro-drugs into their cytotoxic phosphorylated forms. Interestingly, elevated levels of deoxynucleoside kinases are detected in proliferating cells such as cancer cells.

Importantly for viral chemotherapy and some ‘suicide’ gene therapy paradigms, kinases from viruses are found to have broad substrate specificity, phosphorylating a variety of nucleosides analogues. Although no specific information of nucleoside kinase activity in hypoxia is known in the art, reports of over-replication of DNA (Young, D. S. et al Proc Nat Acad Sci USA 85:95533 (1988)) and signalling endothelial cell proliferation (Schafer M. et al FASEB J 17:449 (2003)) in hypoxia implies active DNA synthesis, which likely includes nucleoside phosphorylation as a first metabolic step.

Tables 1-4 disclose a series of new compounds with potential applications in radiosensitization, chemosensitization and chemotherapy of cancer. They are selected so as to undergo selective ‘nucleoside-type’ transport and, most importantly, bioactivation (e.g. phosphorylation) to enhance selective accumulation and promote selective toxicity to hypoxic cells, thereby producing enhanced concentrations in viable but hypoxic cells. Their advantage lies in their improved concentration, and residence half-life, in target cells. Though an exact understanding of the mechanism of action of the present invention is not needed to practise the invention and the present invention is not intended to be limited by any proposed mechanism of action; these characteristics could result because of metabolic trapping as a result of phosphorylation. The compounds disclosed in Tables 1-4 represent azomycin nucleosides that are phosphorylated by nucleoside kinases, and thereby transported by equilibrative, high capacity nucleoside transporters and/or by concentrative nucleoside transporters. Furthermore the halogenated azomycin nucleosides offer optimal lipophilicity (Mannan, R. H. et al J Nucl Med 32:1764 (1991)).

TABLE 1
5, 3 and 2- Halo β-Ribofuranosyl Azomycin and β-Xylofuranosyl Azomycin
derivatives

| | | |
|---|---|---|
| 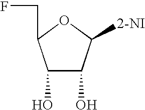 | 5-β-FRAZ | 1-β-D-[5-deoxy-5-fluororibofuranosyl]-2- nitroimidazole |
| 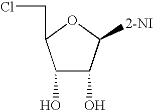 | 5-β-CRAZ | 1-β-D-[5-deoxy-5-chlororibofuranosyl]-2- nitroimidazole |
| 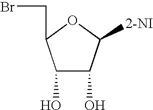 | 5-β-BRAZ | 1-β-D-[5-deoxy-5-bromonbofuranosyl]-2- nitroimidazole |
| 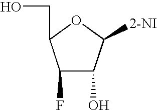 | 3-β-TFRAZ | 1-β-D-[3-deoxy-3-transfluoroxylofuranosyl]-2- nitroimidazole |
| 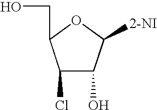 | 3-β-TCRAZ | 1-β-D-[3-deoxy-3-transchloroxylofuranosyl]-2- nitroimidazole |
| 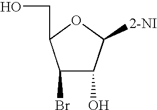 | 3-β-TBRAZ | 1-β-D-[3-deoxy-3-transbromorxylofuranosyl]-2- nitroimidazole |
| 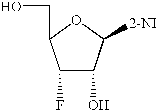 | 3-β-FRAZ | 1-β-D-[3-deoxy-3-fluororibofuranosyl]-2- nitroimidazole |
| 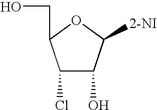 | 3-β-CRAZ | 1-β-D-[3-deoxy-3-chlororibofuranosyl]-2- nitroimidazole |
| 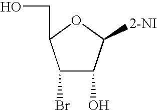 | 3-β-BRAZ | 1-β-D-[3-deoxy-3-bromoribofuranosyl]-2- nitroimidazole |
| 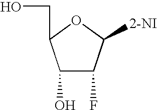 | 2-β-FRAZ | 1-β-D-[2-deoxy-2-fluororibofuranosyl]-2- nitroimidazole |
| 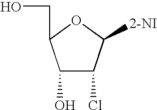 | 2-β-CRAZ | 1-β-D-[2-deoxy-2-chlororibofuranosyl]-2- nitroimidazole |
| 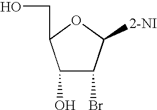 | 2-β-BRAZ | 1-β-D-[2-deoxy-2-bromoribofuranosyl]-2- nitroimidazole |

TABLE 2
5, 3 and 2- Halo β-Arabinofuranosyl Azomycin derivatives

| | | |
|---|---|---|
| 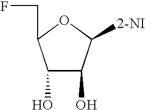 | 5-β-FAZA | 1-β-D-[5-deoxy-5-fluoroarabinofuranosyl]-2- nitroimidazole |
| 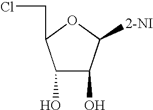 | 5-β-CAZA | 1-β-D-[5-deoxy-5-chloroarabinofuranosyl]-2- nitroimidazole |
| 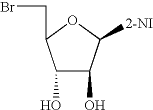 | 5-β-BAZA | 1-β-D-[5-deoxy-5-bromoarabinofuranosyl]-2- nitroimidazole |
| 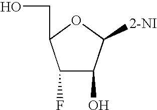 | 3-β-FAZA | 1-β-D-[3-deoxy-3-fluoroarabinofuranosyl]-2- nitroimidazole |
| 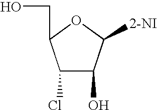 | 3-β-CAZA | 1-β-D-[3-deoxy-3-chloroarabinofuranosyl]-2- nitroimidazole |
| 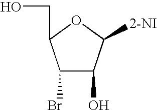 | 3-β-BAZA | 1-β-D-[3-deoxy-3-bromoarabinofuranosyl]-2- nitroimidazole |
| 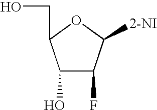 | 2-β-FAZA | 1-β-D-[2-deoxy-2-fluoroarabinofuranosyl]-2- nitroimidazole |
| 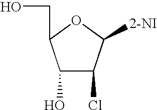 | 2-β-CAZA | 1-β-D-[2-deoxy-2-chloroarabinofuranosyl]-2- nitroimidazole |
| 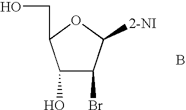 | 2-β-BAZA | 1-β-D-[2-deoxy-2-bromoarabinofuranosyl]-2- nitroimidazole |

TABLE 3
5, 2; 3,2 and 2,3-Dideoxy Halo β-Arabinofuranosyl Azomycin derivatives

| | | |
|---|---|---|
| 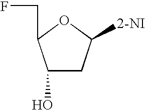 | 5,2-β-DFAZA | 1-β-D-[5,2-dideoxy-5- fluoroarabinofuranosyl]-2- nitroimidazole |
| 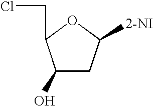 | 5,2-β-DCAZA | 1-β-D-[5,2-dideoxy-5- chloroarabinofuranosyl]-2- nitroimidazole |
| 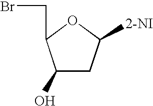 | 5,2-β-DBAZA | 1-β-D-[5,2-dideoxy-5- bromoarabinofuranosyl]-2- nitroimidazole |
| 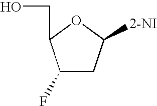 | 52-β-DTFAZA | 1-β-D-[5,2-dideoxy-5- fluoroarabinofuranosyl]-2- nitroimidazole |
| 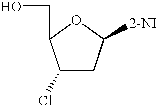 | 5,2-β-DTCAZA | 1-β-D-[5,2-Dideoxy-5-threo- chloroarabinofuranosyl]-2- nitroimidazole |
| 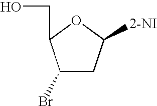 | 5,2-β-DTBAZA | 5,2-Dideoxy-5-threo- bromoarabinofuranosyl]-2- nitroimidazole |
| 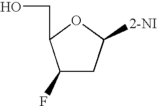 | 3,2-β-DFRAZ | 1-β-D-[3,2-dideoxy-3-fluoroarabinofuranosyl]-2- nitroimidazole |
| 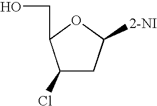 | 3,2-β-DCRAZ | 1-β-D-[3,2-dideoxy-3-chloroarabinofuranosyl]-2- nitroimidazole |
| 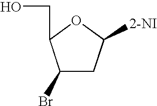 | 3,2-β-DBRAZ | 1-β-D-[3,2-dideoxy-3-bromoarabinofuranosyl]-2- nitroimidazole |
| 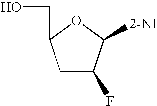 | 32-β-DTFAZ | 1-β-D-[3,2-dldeoxy-3-fluoroarabinofuranosyl]-2- nitroimidazole |
| 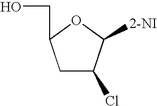 | 3,2-β-DTCAZ | 1-β-D-[3,2-dideoxy-3-chloroarabinofuranosyl]-2- nitroimidazole |
| 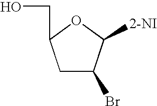 | 3,2-β-DTBAZ | 1-β-D-[3,2-dideoxy-3-bromoarabinofuranosyl]-2- nitroimidazole |
| 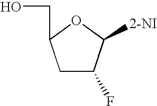 | 2,3-β-DFAZA | 1-β-D-[2,3-dideoxy-2-fluoroarabinofuranosyl]-2- nitroimidazole |
| 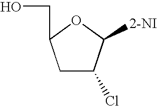 | 2,3-β-DCAZA | 1-β-D-[2,3-dideoxy-2-chloroarabinofuranosyl]-2- nitroimidazole |
| 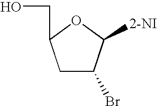 | 2,3-β-DBAZA | 1-β-D-[2,3-dideoxy-2-bromoarabinofuranosyl]-2- nitroimidazole |
| 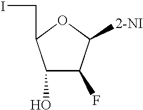 | 2,3-β-DFRAZ | 1-β-D-[2,3-dideoxy-2-fluororibofuranosyl]-2- nitroimidazole |
| 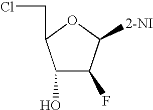 | 2,3-β-DCRAZ | 1-β-D-[2,3-dideoxy-2-chlororibofuranosyl]-2- nitroimidazole |
| 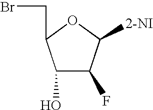 | 2,3-β-DBRAZ | 1-β-D-[2,3-dideoxy-2-bromoribofuranosyl]-2- nitroimidazole |

TABLE 4
Miscellaneous β-Furanosyl Azomycin derivatives

| | | |
|---|---|---|
| 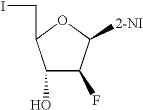 | 5,2-β-IFA | 1-β-D-[2,5-dideoxy-2-fluoro-5- iodoarabinofuranosyl]-2- nitroimidazole |
| 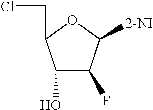 | 5,2-β-CFA | 1-β-D-[2,5-dideoxy-2-fluoro-5- chloroarabinofuranosyl]-2- nitroimidazole |
| 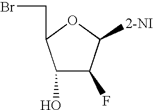 | 5,2-β-BFA | 1-β-D-[2,5-dideoxy-2-fluoro-5- bromoarabinofuranosyl]-2- nitroimidazole |
| 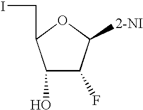 | 5,2-β-IFR | 1-β-D-[2,5-dideoxy-2-fluoro-5- iodoribofuranosyl]-2- nitroimidazole |
| 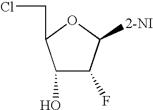 | 5,2-β-CFR | 1-β-D-[2,5-dideoxy-2-fluoro-5- chlororibofuranosyl]-2- nitroimidazole |
| 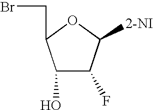 | 5,2-β-BFR | 1-β-D-[2,5-dideoxy-2-fluoro-5- bromoribofuranosyl]-2- nitroimidazole |
| 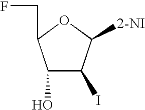 | 2,5-β-IFA | 1-β-D-[2,5-dideoxy-5-fluoro-2- iodoarabinofuranosyl]-2- nitroimidazole |
| 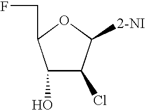 | 2,5-β-CFA | 1-β-D-[2,5-dideoxy-5-fluoro-2- chloroarabinofuranosyl]-2- nitroimidazole |
| 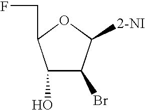 | 2,5-β-BFA | 1-β-D-[2,5-dideoxy-5-fluoro-2- bromoarabinofuranosyl]-2- nitroimidazole |
| 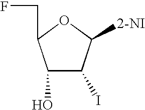 | 2,5-β-IFR | 1-β-D-[2,5-dideoxy-5-fluoro-2- iodoribofuranosyl]-2- nitroimidazole |
| 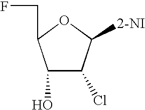 | 2,5-β-CFR | 1-β-D-[2,5-dideoxy-5-fluoro-2- chlororibofuranosyl]-2- nitroimidazole |
| 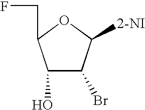 | 2,5-β-BFR | 1-β-D-[2,5-dideoxy-5-fluoro-2- bromoribofuranosyl]-2- nitroimidazole |
| 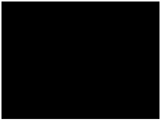 | β-2,3-EFAZ | 1-β-D-[2,3-dideoxy-2,3- epoxyarabinofuranosyl]-2- nitroimidazole |
| 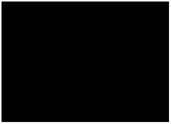 | β-2-KRAZ | 1-β-D-[2-deoxy-2-keto- arabinofuranosyl]-2- nitroimidazole |
| 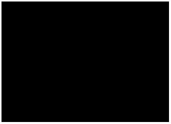 | β-3-KRAZ | 1-β-D-[3-deoxy-3-keto- ribofuranosyl]-2-nitroimidazole |
| 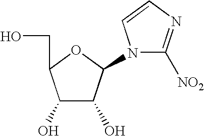 | β-AZR | 1-β-D-[ribofuranosyl]-2- nitroimidazole |
| 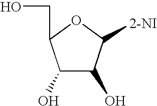 | β-AZA | 1-β-D-[arabinofuranosyl]-2- nitroimidazole |

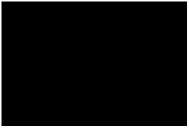

1. All leaving groups at 5′O-
(Ts, Ns, TFMs, Ms, X with
protective groups at 2′ and
3′ when the molecule is
ribose, arabinose, 3′-xylo,
Threo
1-β-D-[2/3/5-
Trisubstitutedfuranosyl]-2-
nitroimidazoles
2. All leaving groups at 3′O-
(Ts, Ns, TFMs, Ms, X with
protective groups at 2′ and
5′ when the molecule is
ribose, arabinose, 3′-xylo,
threo
3. All leaving groups at 2′O-
(Ts, Ns, TFMs, Ms, X with
protective groups at 5′ and
3′ when the molecule is
ribose, arabinose, 3′-xylo,
Threo

The increased transport of the compounds disclosed in Tables 1-4 results in a class of compounds capable of increased residence time, concentration and therefore bioavailability in hypoxic cells, such as tumor cells. The presence of halogens within the compounds allows for inclusion of radioisotopes, the selection of which would be within the ability of one skilled in the art, for radioimaging of hypoxic cells and tissues.

Through inclusion of appropriate radionuclides, the compounds will be appropriate for use in a therapeutic capacity. Examples of appropriate therapeutic radionuclides include the radioiodines ¹²⁵I and ¹³¹I; the radiobromines ⁷⁶Br, ⁷⁷Br, and ⁸²Br; the radiochlorines ^{34m}Cl and ²⁴Cl; and astatine ²¹¹At.

Furthermore, the compounds disclosed in Tables 1-4 are capable of acting as radiosensitizers and chemosensitizers, when administered in association with radio- or chemo-therapy respectively, under conditions known or determinable by those skilled in the art.

Pharmaceutical compositions are also provided, comprising at least one Compound of the Present Invention, and a pharmaceutically acceptable excipient and/or carrier.

The pharmaceutical compositions can be prepared by mixing the desired Compound(s) of the Present Invention with an appropriate vehicle suitable for the intended route of administration. In making the pharmaceutical compositions of this invention, the Compound(s) of the Present Invention are usually mixed with an excipient, diluted by an excipient or enclosed within such a carrier which can be in the form of a capsule, sachet, paper or other container. When the pharmaceutically acceptable excipient serves as a diluent, it can be a solid, semi-solid, or liquid material, which acts as a vehicle, carrier or medium for the therapeutic agent. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the Compounds of the Present Invention, soft and hard gelatin capsules, suppositories, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the Compound(s) of the Present Invention after administration to the patient by employing procedures known in the art.

For preparing solid compositions such as tablets, the Compound(s) of the Present Invention is mixed with a pharmaceutical excipient to form a solid preformulation composition containing a homogeneous mixture of a compound of the present invention. When referring to these preformulation compositions as homogeneous, it is meant that the Compound(s) of the Present Invention are dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the Compound(s) of the Present Invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described herein. The compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

Another formulation employed in the methods of the present invention employs transdermal delivery devices (“patches”). Such transdermal patches may be used to provide continuous or discontinuous infusion of the therapeutic agent of the present invention in controlled amounts. The construction and use of transdermal patches for the delivery of pharmaceutical agents is well known in the art. See, for example, U.S. Pat. No. 5,023,252, herein incorporated by reference. Such patches may be constructed for continuous, pulsatile, or on demand delivery of pharmaceutical agents.

Other suitable formulations for use in the present invention can be found in Remington's Pharmaceutical Sciences.

The following examples are offered to illustrate this invention and are not to be construed in any way as limiting the scope of the present invention.

### EXAMPLE 1

### Preparation of 1-β-D-(Substituted furanosyl/hexopyranosyl)-2-nitroimidazoles

Main synthons 1-β-D-(3,5-O-Tetraisopropyldisilyloxy ribofuranosyl)-2-nitroimidazole and 1-β-D-(2,3-Di-O-acetyl/benzoyl arabinofuranosyl)-2-nitroimidazoles were prepared by the methods described in the literature (Kumar, P. et al Chem Pharm Bull 51:399 (2003); Kumar, P. et al. Tetrahedron Lett 43:4427-4429 (2002)) and were derivatized to develop the compounds claimed in Genus 1. Few compounds under this sub-category are described below.

### 1-β-D-(2-O-Methylthiomethyl-3,5-O-tetraisopropyldisilyloxyribofuranosyl)-2-nitroimidazole

A solution of 1-β-D-(3,5-O-tetraisopropyldisilyloxyribofuranosyl)-2-nitroimidazole (24 mg, 0.05 mmol) in DMSO (0.2 ml) was treated with Ac₂O (0.125 ml) and the mixture was stirred at 22° C. for 2 days. Then 1 ml water was added and extracted with EtOAc and the organic phase was washed with water and dried (Na₂SO₄). After evaporation the residue was chromatographed on silica gel column, eluting with hexanes-ethyl acetate (10:1) to give 1-β-D-(2-O-methylthiomethyl-3,5-O-tetraisopropyldisilyloxy ribofuranosyl)-2-nitroimidazole (10 mg, 37%) as a viscous oil. ¹H-NMR, MS.

### 1-β-D-(5-O-Acetylribofuranosyl)-2-nitroimidazoIe

A solution of 1-β-D-(2,3,5-tri-O-acetylribofuranosyl)-2-nitroimidazole (37.1 mg, 0.1 mmol) (Naimi, E. et al Nucleosides Nucleotides Nucleic Acids 24:173 (2005)) and N₂H₂ (12.8 mg, 0.4 mmol) in glacial acetic acid-pyridine (1:4, 1 ml) was heated at 80° C. for 3 h. After quenching with acetone (0.5 ml) and stirring at 22° C. for 1 h, the solvents were evaporated and the residue was purified on silica gel column, using ethyl acetate-hexanes (80:20, v/v) to give 1-β-D-(5-O-acetylribofuranosyl)-2-nitroimidazole (20 mg, 70%); ¹H-NMR, HRMS.

### 1-β-D-(3,5-Di-O-acetyl ribofuranosyl)-2-nitroimidazole and 1-β-D-(2,5-Di-O-acetyl ribofuranosyl)-2-nitroimidazole

A solution of 1-β-D-(2,3,5-tri-O-acetylribofuranosyl)-2-nitroimidazole (9.3 mg, 0.025 mmol) and N₂H₂ (1.2 mg, 0.037 mmol) in glacial acetic acid-pyridine (1:4, 0.25 ml) was stored at 22° C. for 9 h. After quenching with acetone, the solvents were evaporated and the residue was purified on preparative TLC, CHCl₃-MeOH (9:5, v/v) to give mixture of 1-β-D-(3,5-di-O-acetylribofuranosyl)-2-nitroimidazole and 1-β-D-(2,5-di-O-acetylribofuranosyl)-2-nitroimidazole; ¹H-NMR, and trace of 1-β-D-(5-O-acetylribofuranosyl)-2-nitroimidazole.

### 1-β-D-(3-O-p-Toluenesulfonyl ribofuranosyl)-2-nitroimidazole and 1-β-D-(2-O-p-Toluenesulfonyl ribofuranosyl)-2-nitroimidazole

To a stirred suspension of 1-β-D-(ribofuranosyl)-2-nitroimidazole (49 mg, 0.20 mmol) in CH₃CN (6 ml) was added Bu₂SnO (56 mg, 0.225 mmol), p-toluenesulfonyl chloride (64 mg, 0.335 mmol) and TBAF in THF (1.0 M solution, 0.2 ml, 0.20 mmol) at 22° C. After 24 h stirring, another portion of p-toluenesulfonyl chloride (38 mg, 0.20 mmol) was added and stirring continued overnight. The solvent was evaporated and the residue was chromatographed on silica gel column, using dichloromethane-ethyl acetate (70:30) to give 1-β-D-(3-O-p-toluenesulfonyl ribofuranosyl)-2-nitroimidazole (19 mg, 24%); m.p. 172-173° C.; ¹H-NMR, HRMS, and 1-β-D-(2-O-p-toluenesulfonyl ribofuranosyl)-2-nitroimidazole (31 mg, 39%); m.p. 165-166° C., ¹H-NMR, HRMS.

### 1-β-D-(3,5-O-Tetraisopropyldisilyloxy-2-O-p-toluenesulfonylribofuranosyl)-2-nitroimidazole

A mixture of 1-β-D-(3,5-O-Tetraisopropyldisilyloxyribofuranosyl)-2-nitroimidazole (48.7 mg, 0.1 mmol), p-toluenesulfonyl chloride (95.3 mg, 0.5 mmol) and DMAP (6.1 mg, 0.05 mmol) in dry pyridine (1 ml) was stirred at 50-55° C. overnight. Another portion of p-toluenesulfonyl chloride (78.6 mg, 0.4 mmol) and DMAP (4.9 mg, 0.04 mmol) was added and stirring at 50-55° C. continued for 12-14 h. After evaporation of solvent and purification on silica gel column, using hexanes-ethyl acetate (87.5:12.5, v/v), gave 1-β-D-(3,5-(-tetraisopropyldisilyloxy-2-O-p-toluenesulfonylribofuranosyl)-2-nitroimidazole (49 mg, 75%); m.p. 146-147° C., ¹H-NMR, mass, HRMS.

### 1-β-D-3,5-Di-O-acetyl-2-O-p-toluenesulfonylribofuranosyl)-2-nitroimidazole

Ac₂O (0.045 ml) was added to a solution of 1-(2-O-p-toluenesulfonyl-β-D-ribofuranosyl)-2-nitroimidazole (24 mg, 0.06 mmol) in anhydrous pyridine and the mixture was stirred at 22° C. overnight. The solvent was removed and the residue was purified on silica gel column, using hexanes-ethyl acetate (50:50) to give 1-(3,5-Di-O-acetyl-2-O-p-toluenesulfonyl-β-D-ribofuranosyl)-2-nitroimidazole (26 mg, 90%).

### 1-β-D-(5-O-tert-Butyldiphenylsilyl-2,3-di-O-acetyl ribofuranosyl)-2-nitroimidazole and 1-β-D-(3,5-Di-O-tert-butyldiphenylsilyl-2-O-acetyl ribofuranosyl)-2-nitroimidazole

1-β-D-(ribofuranosyl)-2-nitroimidazole (270 mg, 1.1 mmol) was dissolved in dry pyridine (1.25 ml), and tert-butyldiphenylsilyl chloride (316 mg, 1.15 mmol) was added. The reaction mixture was stirred at 22° C. overnight. Additional tert-butyldiphenylsilyl chloride (32 mg) was added and after completion, Ac₂O (0.415 ml, 4.0 mmol) was added and stirred overnight. After solvent evaporation, the residue was purified on column, eluting with hexanes-ethyl acetate (70:30) to give 1-β-D-(5-O-tert-Butyldiphenylsilyl-2,3-di-O-acetylribofuranosyl)-2-nitroimidazole (478 mg, 77%); m.p 52-53° C., ¹H-NMR, ¹³C-NMR, HRMS, and 1-(3,5-Di-O-tert-butyldiphenylsilyl-2-O-acetyl-β-D-ribofuranosyl)-2-nitroimidazole (33 mg, 4%) as a viscous oil; ¹H-NMR, HRMS.

### 1-β-D-(2,3-Di-O-acetyl ribofuranosyl)-2-nitroimidazole

A suspension of 1-β-D-(5-O-tert-Butyldiphenylsilyl-2,3-di-O-acetyl ribofuranosyl)-2-nitroimidazole (454 mg, 0.8 mmol), benzoic acid (683 mg, 5.6 mmol) and KF (325 mg, 5.6 mmol) in MeCN (20 ml) was heated at 75-80° C. for 16 h. After cooling and filtration, the filtrate was evaporated and the residue was chromatographed on silica gel column, eluted with ethyl acetate-hexanes (70:30) to afford 1-β-D-(2,3-Di-O-acetylribofuranosyl)-2-nitroimidazole (246 mg, 93%); m.p. 122-123° C.; ¹H-NMR, ¹³C-NMR, HRMS.

### 1-β-D-(2,3-Di-O-acetyl-5-O-toluenesulfonyl arabinofuranosyl)-2-nitroimidazole

A solution of toluenesulfonyl chloride (0.23 g, 1.2 mmol) in anhydrous pyridine (5 mL) was added to a pre-cooled solution of 1-β-D-(2-O-acetylarabinofuranosyl)-2-nitroimidazole (0.23 g, 0.8 mmol) in anhydrous pyridine (20 mL) and stirred at 0° C. for 18 h. Once the starting precursor was completely consumed (determined by tlc examination), acetic anhydride (1.2 mmol) was added to this mixture and the stirring was continued for an additional 3 h. The solvent was evaporated after the reaction was complete and the mixture was purified on a silica gel column using hexanes/ethyl acetate (1:1, v/v) as eluent, m.p. 117-119° C., ¹H-NMR, ¹³C-NMR.

### EXAMPLE 2

### Preparation of 1-β-D-[(2/3/5-Substituted) or (2,3-disusbstituted) or (2,2-disubstituted) or (3/3-disubstituted) or (2,5-disubstituted) or (3,5-disubstituted) furanosyl/hexopyranosyl)-2-nitroimidazoles.

### 1-β-D-(2-Deuterio-3,5-O-tetraisopropyldisilyloxyarabinofuranosyl)-2-nitroimidazole

A stirred suspension of CrO₃ (15 mg) in CH₂Cl₂ (1 ml) was cooled to 0° C. and Ac₂O (0.015 ml) and pyridine (0.025 ml) were added. After 3 min, 1-β-D-(3,5-O-tetraisopropyldisilyloxy ribofuranosyl)-2-nitroimidazole (24 mg, 0.05 mmol) was added and then allowed to warm to 5-10° C. over a period of 2 h. Volatile materials were evaporated and the residue was cooled to 0° C. and dissolved in absolute EtOH (1.0 ml). The stirred mixture was treated by addition of NaBD₄ (3 mg). After 30 min a second portion of NaBD₄ (3 mg) was added and a 2 mg portion was added at 1 h and allowed to warm to 10-12° C. and stirred for 30 min. After evaporation, the residue was chromatographed on a silica column, eluting with hexanes-ethyl acetate (80:20) to afford 1-β-D-(2-deuterio-3,5-O-tetraisopropyldisilyloxy arabinofuranosyl)-2-nitroimidazole (15 mg, 61%); m.p. 160-161° C., ¹H-NMR, ¹³C-NMR, mass, HRMS.

### 1-β-D-(2-Deuterio-arabinofuranosyl)-2-nitroimidazole

A suspension of KF (41 mg, 0.7 mmol), benzoic acid (85 mg, 0.7 mmol) and 1-β-D-(2-Deuterio-3,5-O-tetraisopropyldisilyloxy arabinofuranosyl)-2-nitroimidazole (58 mg, 0.12 mmol) in MeCN (8 ml) was heated at 75° C. for 3.5 h. After cooling and filtration, the filtrate was evaporated and the residue was purified by column chromatography, using ethyl acetate as a eluent to give 1-β-D-(2-Deuterio arabinofuranosyl)-2-nitroimidazole (23 mg, 78%); m.p. 163-164° C., ¹H-NMR, ¹³C-NMR, HRMS.

### 1-β-D-(2-Deuterio-5-deoxy-5-fluoroarabinofuranosyl)-2-nitroimidazole

1-β-D-(2-Deuterio arabinofuranosyl)-2-nitroimidazole (20 mg, 0.08 mmol) was dissolved in anhydrous DME (6 ml) and cooled to −10° C. and DAST (14 mg, 0.0.09 mmol) was added. After 1 h, the reaction mixture was allowed to warm to 0° C. and after 2.5 h additional DAST (20 mg, 0.12 mmol) was added in three portions every 1 h and stirred for additional 5 h at 0° C. Additional DAST (10 mg, 0.06 mmol) was added until starting material disappeared on TLC. After quenching with MeOH and solvent evaporation, the products were separated by preparative TLC gave 1-β-D-(2-deuterio-5-deoxy-5-fluoroarabinofuranosyl)-2-nitroimidazole (2 mg); ¹H-NMR, ¹⁹F-NMR, MS.

### 1-β-D-(2,3-Di-O-acetyl-5-deoxy-5-fluoroarabinofuranosyl)-2-nitroimidazole

DAST (0.613 g, 3.8 mmol) was added to a solution of 1-β-D-(2,3-Di-O-acetylarabinofuranosyl)-2-nitroimidazole in CH₂Cl₂ (9 mL) and pyridine (1 mL) at −78° C. and stirred at this temperature for 8 h. The temperature was warmed up to 22° C. and the stirring was continued for an additional 16 h. Afterwards, the reaction mixture was cooled down to ° C. and quenched by adding ice water to it. Column chromatographic purification of the mixture, after removal of the solvents, using hexanes/ethyl acetate (1:1, v/v) afforded pure 1-β-D-(2,3-Di-O-acetyl-5-deoxy-5-fluoroarabinofuranosyl)-2-nitroimidazole. m.p. 112-114° C., ¹H NMR, ¹³C NMR, ¹⁹F-NMR.

### 1-β-D-(5-Deoxy-5-fluoroarabinofuranosyl)-2-nitroimidazole

Treatment of 1-β-D-(2,3-Di-O-acetyl-5-deoxy-5-fluoroarabinofuranosyl)-2-nitroimidazole with 2M. NH3/MeOH solution at 22° C. afforded 1-(-β-D-5-Deoxy-5-fluoroarabinofuranosyl)-2-nitroimidazole which was purified by column chromatography using ethyl acetate as an eluent, ¹H NMR, ¹³C NMR, ¹⁹F NMR.

### 1-β-D-(2,3-Di-O-acetyl-5-deoxy-5-chloroarabinofuranosyl)-2-nitroimidazole

Trifluoromethanesulfonyl fluoride (0.53 g, 3.19 mmol) was added to a pre-cooled stirred solution (−80° C.) of 1-β-D-(2,3-Di-O-acetyl arabinofuranosyl)-2-nitroimidazole (0.55 g, 1.67 mmol) and dimethylamino pyridine (0.62g, 5 mmol) in anhydrous dichloromethane (40 mL) and the reaction was continued for 2 h. Afterwards the temperature of the reaction was raised to 22° C. and, then, quenched with water. The solvent was removed from the eraction mixture and the product was chromatographed on a silica gel column using hexanes/ethyl acetate (40/60, v/v) to afford this product. m.p. 128° C., ¹H NMR, HRMS, CHN.

### 1-β-D-(3,5-Di-O-benzoyl-2-deoxy-2-fluoroarabinofuranosyl)-2-nitroimidazole and 1-β-D-(2,5-di-O-benzoyl-3-deoxy-3-fluorolyxofuranosyl)-2-nitroimidazole

DAST (280 mg, 1.8 mmol) was added to a solution of 1-β-D-(3,5-Di-O-benzoylarabinofuranosyl)-2-nitroimidazole (32) and 1-β-D-(2,5-di-O-benzoylribofuranosyl)-2-nitroimidazole mixture (163 mg, 0.36 mmol) in pyridine (0.24 ml) and CH₂Cl₂ (10 ml) under ice bath, and stirred at 0-5° C. for 1 h and then allowed to warm to 22° C. After 7 h, second portion of DAST (140 mg, 0.9 mmol) was added and stirring continued at same temperature for an additional 14 h. Then, the reaction mixture was quenched by addition of MeOH, the solvents were evaporated and the residue was purified by preparative thin layer chromatography, using CHCl₃ as a solvent to afford 1-β-D-(3,5-di-O-benzoyl-2-deoxy-2-fluoroarabinofuranosyl)-2-nitroimidazole (67 mg, 41%); m.p 162-163° C., ¹H-NMR, ¹³C-NMR, ¹⁹F-NMR, MS, HR-mass, and 1-β-D-(2,5-di-O-benzoyl-3-deoxy-3-fluorolyxofuranosyl)-2-nitroimidazole (35 mg, 21%) as a viscous oil; ¹H-NMR, ¹³C-NMR, ¹⁹F-NMR, MS.

### 1-β-D-(2-Deoxy-2-fluoroarabinofuranosyl)-2-nitroimidazole

A solution of 1-β-D-(3,5-di-O-benzoyl-2-deoxy-2-fluoroarabinofuranosyl)-2-nitroimidazole (55 mg, 0.12 mmol) in methanolic ammonia (4 ml, 2.0 M) was stirred at 22° C. for 9.5 h. After evaporation of solvent, the residue was chromatographed on silica gel column, eluting with CHCl₃-MeOH (92.5:7.5) to give 1-β-D-(2-Deoxy-2-fluoroarabinofuranosyl)-2-nitroimidazole (27 mg, 92%); m.p 183-185° C., ¹H-NMR, ¹³C-NMR, ¹⁹F-NMR.

### 1-β-D-(3-Deoxy-3-fluorolyxofuranosyl)-2-nitroimidazole

A solution of 1-β-D-(2,5-di-O-benzoyl-3-deoxy-3-fluorolyxofuranosyl)-2-nitroimidazole (30 mg) in methanolic ammonia (3 ml, 2.0 M) was stirred at 22° C. for 14 h. After evaporation of solvent, the residue was chromatographed on silica gel column to give 1-β-D-(3-deoxy-3-fluorolyxofuranosyl)-2-nitroimidazole (16 mg) with minor impurity; ¹H-NMR, ¹⁹F-NMR.

### 1-β-D-(5-Deoxy-5-fluororibofuranosyl)-2-nitroimidazole

To a solution of 1-β-D-(2,3-Di-O-acetylribofuranosyl)-2-nitroimidazole (228 mg, 0.69 mmol) and pyridine (0.51 ml) in CH₂Cl₂ at −20° C., DAST was added and then allowed to warm to 0° C. 0ver a period of 3 h. The reaction solution was stirred at 0° C. for 12 h. After addition of DAST (0.09 ml, 0.69 mmol), stirring was continued at 0° C. for 12 h but not completed so additional DAST (0.09 ml, 0.69 mmol) was added and stirred at 5° C. for 12 h to completion. Then, the reaction solution was quenched with MeOH and evaporated. NH₃/MeOH (2.9 M, 15 ml) was added and stirred at 5° C. overnight. After evaporation of solvent, the residue was chromatographed on silica gel column, using CH₂Cl₂-MeOH (96:4, v/v) to afford 1-β-D-(5-deoxy-5-fluororibofuranosyl)-2-nitroimidazole (75 mg, 44%); m.p 151-153° C; ¹H-NMR, ¹³C-NMR, ¹⁹F-NMR, mass, HRMS.

### EXAMPLE 3

### Preparation of 1-β-D-[(2/3-Epoxy)-5-susbstitutedfuranosyl/hexopyranosylsyl)-2-nitroimidazoles

### 1-β-D-[(2/3-Epoxy)-5-deoxy-5-fluorofuranosyl/hexosyl)-2-nitroimidazole

This product was obtained as a side product during the synthesis of 1-β-D-(5-deoxy-5-fluoroarabinofuranosyl)-2-nitroimidazole. It was isolated and characterized by ¹H-NMR, ¹⁹F-NMR, MS.

### 1-β-D-[(2/3-Epoxy)-2-deutero-5-deoxy-5-fluorofuranosyl/hexosyl)-2-nitroimidazole

This product was obtained as a side product during the synthesis of 1-β-D-(2-deuterio-5-deoxy-5-fluoroarabinofuranosyl)-2-nitroimidazole. It was isolated and characterized by ¹H-NMR, ¹⁹F-NMR, MS.

### EXAMPLE 4

### Preparation of 1-α-D-[(2/3-Substituted) or (2,3-disusbstituted) or (2,2-disubstituted) or (3/3-disubstituted) furanosyl/hexopyranosyl)-2-nitroimidazoles

### 1-α-D-(3,5-Di-O-benzoyl-arabinofuranosyl)-2-nitroimidazole

1-α-D-(2,3,5-Tri-O-benzoylarabinofuranosyl)-2-nitroimidazole (REF) was dissolved in anhydrous terahydrofuran (THF, 6 mL) and chilled to −56° C. 1M solution of potassium tert-butoxide in THF (6.1 mL) was added to this solution under sitrring in an inert atmosphere. After the reaction was complete (15 min), the mixture was quenched by adding DOWEX-50™ until the pH was 7. The resin was filtered and the filtrate was subjected to solvent removal by evaporation. Column chromatography of this mixture on a silica gel column using hexanes/ethyl acetate (1:3, v/v) and, then recrystallization of purified product in hexanes/ethylacetate (3:1, /v/v, 6 mL) afforded pure 1-α-D-(3,5-Di-O-benzoylarabinofuranosyl)-2-nitroimidazole. m.p. 194-196° C., ¹H NMR, ¹³C NMR.

### 1-α-D-(3,5-Di-O-benzoyl-2-O-[toluene/p-nitrobenzene]sulfonylribofuranosyl)-2-nitroimidazole

A mixture of 1-α-D-(3,5-di-O-benzoylribofuranosyl)-2-nitroimidazole (0.2 g, 0.44 mmol), toluenesulfonyl chloride or p-nitrobenzenesulfonyl chloride (1.32 mmol) an DMAP (0.16 g, 1.32 mmol) was taken in anhydrous pyridine (15 mL) and stirred at 45-50° C. under argon for 16 h and, then, the solvent was removed by evaporation. Purification of this mixture on a silica gel column using hexanes/ethyl acetate (2:1, v/v) gave this product. m.p. 60-61° C., ¹H NMR, ¹³C NMR, CHN.

### 1-α-D-(3,5-Di-O-benzoyl-2-deoxy-2-fluororibofuranosyl)-2-nitroimidazole

1-α-D-(3,5-Di-O-benzoyl-arabinofuranosyl)-2-nitroiidazole (0.6 g, 1.32 mmol) was treated with DAST (1.07 g, 6.6 mmol) in anhydrous dichloromethane (10 mL) and pyridine (0.2 mmol) at 0° C. for 4 h. Work up and purification of this product was done as described for other fluorinated compounds in this patent application. m.p. 56-57° C., ¹H NMR, ¹³C NMR, ¹⁹F NMR, CHN.

### 1-α-D-(2-Deoxy-2-Fluororibofuranosyl)-2-nitroimidazole

1-(3,5-Di-O-benzoyl-2-deoxy-2-fluoro-α-D-ribofuranosyl)-2-nitroimidazole (50 mg) was dissolved in NH₃/MeOH (2.0 M, 8 ml) and stirred overnight at 5° C. then the solvent was removed and the residue was purified on a silica gel column using MeOH-CHCl₃ (7:93) to give 1-α-D-(2-deoxy-2-fluororibofuranosyl)-2-nitroimidazole (20 mg).m.p. 155-157° C., ¹H NMR, ¹³C NMR, ⁹F NMR, CHN.

### 1-(2-Deuterio-3,5-O-tetraisopropyldisilyloxy-α-D-ribofuranosyl)-2-nitroimidazole

A stirred suspension of CrO₃ (15 mg) in CH₂Cl₂ (1 ml) was cooled to 0° C. and Ac₂O (0.015 ml) and pyridine (0.025 ml) were added. After 3 min, 1-α-D-(3,5-O-O-tetraisopropyldisilyloxyarabinofuranosyl)-2-nitroimidazole (3,5-TIPS-α-AZA, (24mg, 0.05 mmol) was added to this solution and then allowed to warm to 5-10° C. over a period of 2 h. Volatile materials were evaporated and the residue was cooled to 0° C. and dissolved in absolute EtOH (1.0 ml). The stirred mixture was treated by addition of NaBD₄ (3 mg). After 30 min a second portion of NaBD₄ (3 mg) was added and a 2 mg portion was added at 1 h and allowed to warm to 10-12° C. and stirred for 30 min. After evaporation, the residue was chromatographed on a silica column, eluting with hexanes-ethyl acetate (80:20, v/v) to afford 1-α-D-(2-deuterio-3,5-O,O-tetraisopropyldisilyloxy ribofuranosyl)-2-nitroimidazole (15 mg, 61%); m.p 83-84° C., ¹H-NMR, ³C-NMR, HISS.

### 1-α-D-(2-Deuterio ribofuranosyl)-2-nitroimidazole

A stirred suspension of potassium fluoride (29 mg, 0.5 mmol), benzoic acid (61 mg, 0.5 mmol) and 1-α-D-(2-deuterio-3,5-O,O-tetraisopropyldisilyloxyribofuranosyl)-2-nitroimidazole (41 mg, 0.084 mmol) in CH₃CN (5 ml) was heated to 75° C. After the reaction was over, the mixture was cooled, filtered and the filtrate was evaporated over a rotavapor. The residue was purified by column chromatography using MeOH:CHCl₃ (8:92, v/v) to afford pure product. ¹H-NMR, ¹³C-NMR.

### EXAMPLE 5

### Preparation of 1-α-D-[(2/3-Epoxy)-5-susbstituted furanosyl/hexopyranosyl)-2-nitroimidazoles

### 1-α-D-(2,3-Epoxy-5-deoxy-5-fluororibofuranosyl)-2-nitroimidazole

DAST (0.5 mmol) was added to a cooled (0° C.) suspension of A-AZA (0.1 mmol) in anhydrous DME and the stirring was continued at this temperature for 8 h. After quenching with MeOH, the solvents were removed and 1-α-D-(2,3-epoxy-5-fluoro-ribofuranosyl)-2-nitroimidazole was isolated by chromatography. ¹H-NMR, ¹⁹F-NMR

### EXAMPLE 6

### Radiochemical Synthesis

³H, ¹⁸F, ^{75/76/77}Br, ^{34m/34/36}Cl, ¹²⁵I, ¹²³I, ¹³¹I, ¹²⁴I and ²¹¹At labeled analogs of the products described under two genii and their labeling procedures are claimed under this patent. The details of general synthesis procedures for isotopic labeling of the Compounds of the Present Invention are described below.

Tritiation/Deuteration:

A stirred suspension of CrO₃ (15 mg) in CH₂Cl₂ (1 ml) was cooled to 0° C. and Ac₂O (0.015 ml) and pyridine (0.025 ml) were added. After 3 min, 1-β-D-(3,5-O-tetraisopropyldisilyloxy arabinofuranosyl)-2-nitroimidazole (24 mg, 0.05 mmol) is added and then allowed to warm to 5-10° C. over a period of 2 h. Volatile materials were evaporated and the residue was cooled to 0° C. and dissolved in absolute EtOH (1.0 ml). The stirred mixture is treated by addition of NaBT₄ (3 mg). After 30 min a second portion of NaBT₄ (3 mg) was added and a 2 mg portion was added at 1 h and allowed to warm to 10-12° C. and stirred for 30 min. After evaporation, the residue is chromatographed on a silica column, eluting with hexanes-ethyl acetate (80:20) to afford corresponding tritiated product.

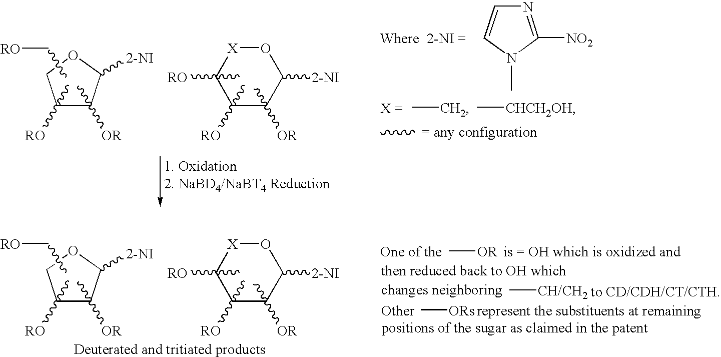

Similarly, the syntheses of other tritiated Compounds of the Present Invention may be undertaken.

Radiohalogenation: ¹⁸F, ^{75/76/77}Br, ^{34m/34/36}Cl, ¹²⁵I, ¹²³I, ¹³¹I, ¹²⁴I and ²¹¹At labeled analogs of the Compounds of the Present Invention. Radiofluorination process with ¹⁸F isotope is provided as an illustrative example of radiohalogenation, though one skilled in the art would recognize that other radiohalogens could be utilised.

Radiofluorination of the Compounds of the Present Invention is carried out by using three radiofluorinated reagents namely K-2.2.2/¹⁸F/K₂CO₃ complex, ¹⁸F-DAST and R₄N[¹⁸F]F.

The precursors for radio(fluorin)halogention, pre-dissolved in appropriate solvent are allowed to react with appropriate radiofluorination reagent (K-2.2.2/¹⁸F/K₂CO₃ complex, ¹⁸F-DAST and R₄N[¹⁸F]F) in an inert atmosphere. This process is temperature and time specific for radiohalogenation of every precursor. This is followed by removal of the protective groups and purification (automated or designed HPLC chromatography) to afford pure radiofluorinated product.

The radiofluorination process is outlined below:

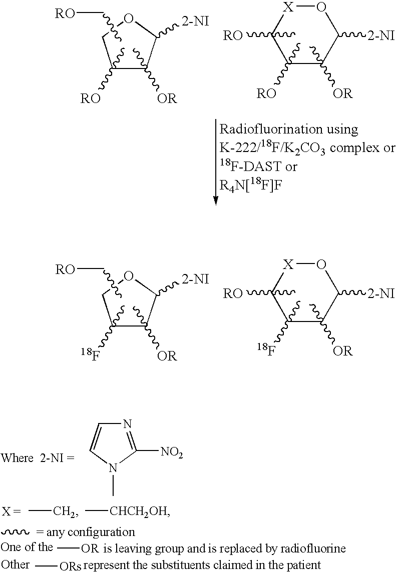

The products radiofluorinated using this process include 1-α-D-(2-deoxy-2-fluoro-ribofuranosyl)-2-nitroimidazole, 1-β-D-(5-deoxy-5-fluoroarabinofuranosyl)-2-nitroimidazole and 1-β-D-(2-deoxy-2-fluororibofuranosyl)-2-nitroimidazole

Although the disclosure describes and illustrates various embodiments of the invention, it is to be understood that the invention is not limited to these particular embodiments. Many variations and modifications will now occurr to those skilled in the art.

### EXAMPLE 7

### Use of Compounds as Sensitizers in Cells Under Hypoxic Conditions

The human colorectal carcinoma cell line HCT116 (WT) was used to observe the effects of selected Compounds of the Present Invention in sensitizing the cells, under conditions of hypoxia, to radiotherapy. All hypoxia sensitizers with the exception of β-3-FTAZR, which was dissolved in DMSO, were dissolved in 95% ethanol to the concentration of 10 mM. Cell treatment was performed for 30 min. prior to degassing and irradiation at 100 μM concentration of the tested sensitizer. Cells were irradiated in 60Co γ-irradiator at doses: 4-8-12-16 & 20 Gy.

Approximately 300,000 cells were seeded per T60 glass dish with 3 ml DMEM/F12 media added per dish. Dishes were incubated in 5% CO₂ at 37° C. overnight. On the second day media in each dish was replaced with 2 ml fresh DMEM/F12. The plates were degassed in nitrogen in 6 groups of 2 dishes per chamber. Dishes were incubated for 30 min. on oscillating shaker at R/T X 60 cycles per min and irradiated as follows:
N2 chamber/2 dishes at 0 Gy (Control)
N2 chamber/2 dishes at 4 Gy
N2 chamber/2 dishes at 8 Gy
N2 chamber/2 dishes at 12 Gy
N2 chamber/2 dishes at 16 Gy
N2 chamber/2 dishes at 20 Gy
Air chamber/2 dishes at 0 Gy (Control)
Air chamber/2 dishes at 4 Gy
Air chamber/2 dishes at 8 Gy
Air chamber/2 dishes at 12 Gy
Air chamber/2 dishes at 16 Gy
Air chamber/2 dishes at 20 Gy

Cells were trypsinized from each dish and plate them at density, from 100 to 15000 cells/5 ml media for oxic conditions and 100 & 5000 cells/5 ml media for hypoxic conditions. Media was decanted from the dish, washed twice with PBS and 500 μl of Trypsin added. Trypsinization was quenched with 4.5 ml fresh media and serial dilutions of cell cells were made as follows: (A) 1:10; (B) 1:100; (C) 1:1000. To each dish was added 1000 μl of dilution (C) for 100 cells per dish or 100 μl of dilution (B) for 100 cells per dish. Cells were incubated 10 to 14 days at 5% CO₂ at 37° C. On Day 10 to 14 colonies were stained with Methylene Blue or Crystal Violet stain in EtOH. Colonies were counted and plotted accordingly.

As can be seen in FIG. 2, use of the selected compound of the present invention decreased survival of cells under hypoxic conditions, compared to those cells under hypoxic conditions not similarly treated.

As shown in FIG. 3 Compounds of the Present Invention, in particular LAZA compounds, can be utilized to effect a cytotoxicity in a cell population.

While particular embodiments of the present invention have been described in the foregoing, it is to be understood that other embodiments are possible within the scope of the invention and are intended to be included herein. It will be clear to any person skilled in the art that modifications of and adjustments to this invention, not shown, are possible without departing from the spirit of the invention as demonstrated through the exemplary embodiments. The invention is therefore to be considered limited solely by the scope of the appended claims.

What is claimed is:

1. A compound useful for the therapeutic killing of hypoxic cells in a patient, said compound comprising the general structure of a 1-β-D-(Substituted pentosyl/hexosyl)-2-nitroimidazoles or 1-α-D-(Substituted furanosyl/hexopyranosyl)-2-nitroimidazoles.

2. A compound useful for therapeutic treatment of hypoxic cells in a patient, said compound selected from the group consisting of:
1-β-D-[3-deoxy-3-fluoroxylofuranosyl]-2-nitroimidazole,
1-β-D-[3-deoxy-3chloroxylofuranosyl]-2-nitroimidazole,
1-β-D-[3-deoxy-3-bromoroxylofuranosyl]-2-nitroimidazole,
1-β-D-[3-deoxy-3-fluororibofuranosyl]-2-nitroimidazole,
1-β-D-[3-deoxy-3-chlororibofuranosyl]-2-nitroimidazole,
1-β-D-[3-deoxy-3-bromoribofuranosyl]-2-nitroimidazole,
1-β-D-[2-deoxy-2-fluororibofuranosyl]-2-nitroimidazole,
1-β-D-[2-deoxy-2-chlororibofuranosyl]-2-nitroimidazole,
1-β-D-[2-deoxy-2-bromoribofuranosyl]-2-nitroimidazole,
1-β-D-[3-deoxy-3-fluoroarabinofuranosyl]-2-nitroimidazole,
1-β-D-[3-deoxy-3-chloroarabinofuranosyl]-2-nitroimidazole,
1-β-D-[3-deoxy-3-bromoarabinofuranosyl]-2-nitroimidazole,
1-β-D-[2-deoxy-2-fluoroarabinofuranosyl]-2-nitroimidazole,
1-β-D-[2-deoxy-2-chloroarabinofuranosyl]-2-nitroimidazole,
1-β-D-[2-deoxy-2-bromoarabinofuranosyl]-2-nitroimidazole,
1-β-D-[3,2-dideoxy-3-fluoroarabinofuranosyl]-2-nitroimidazole,
1-β-D-[3,2-dideoxy-3-chloroarabinofuranosyl]-2-nitroimidazole,
1-β-D-[3,2-dideoxy-3-bromoarabinofuranosyl]-2-nitroimidazole,
1-β-D-[3,2-dideoxy-3-fluoroarabinofuranosyl]-2-nitroimidazole,
1-β-D-[3,2-dideoxy-3-chloroarabinofuranosyl]-2-nitroimidazole,
1-β-D-[3,2-dideoxy-3-bromoarabinofuranosyl]-2-nitroimidazole,
1-β-D-[2,3-dideoxy-2-fluoroarabinofuranosyl]-2-nitroimidazole,
1-β-D-[2,3-dideoxy-2-chloroarabinofuranosyl]-2-nitroimidazole,
1-β-D-[2,3-dideoxy-2-bromoarabinofuranosyl]-2-nitroimidazole,
1-β-D-[2,3-dideoxy-2-fluororibofuranosyl]-2-nitroimidazole,
1-β-D-[2,3-dideoxy-2-chlororibofuranosyl]-2-nitroimidazole,
1-β-D-[2,3-dideoxy-2-bromoribofuranosyl]-2-nitroimidazole,
1-β-D-[2-deoxy-2-keto-arabinofuranosyl]-2-nitroimidazole,
1-β-D-[3-deoxy-3-keto-arabinofuranosyl]-2-nitroimidazole, and
1-β-D-[2,3-dideoxy-2,3-epoxyarabinofuranosyl]-2-nitroimidazole.
